(19) United States
(12) Patent Application Publication
(10) Pub. No.: US 2026/0088145 A1
(43) Pub. Date: Mar. 26, 2026

(54) HEALTHCARE MANAGEMENT SYSTEM

(71) Applicant: ORTHO MARKETING, LLC, Draper, UT (US)

(72) Inventors: Marc E. Jackson, Draper, UT (US); Jackson M. Neeley, Draper, UT (US)

(21) Appl. No.: 19/339,110

(22) Filed: Sep. 24, 2025

Related U.S. Application Data
(60) Provisional application No. 63/698,930, filed on Sep. 25, 2024.
(60) Provisional application No. 63/750,419, filed on Jan. 28, 2025.

(51) Int. Cl. G16H 15/00; G16H 10/20; G16H 10/60; G16H 50/20; G16H 50/70

(52) U.S. Cl. CPC G16H 15/00; G16H 10/20; G16H 10/60; G16H 50/20; G16H 50/70

(57) ABSTRACT

A computer system for structured analysis of medical reports includes one or more processors and computer-readable media storing instructions that configure the system to process user-supplied text and medical report data. Written text, including linguistic variants and descriptive phrases associated with injury categories, is received and vectorized into a first embedding vector. The text and embedding vector are stored in a hybrid database comprising predefined labels corresponding to injury categories and severity codes, free-form text fields storing linguistic variants and descriptive phrases, and stored vector embeddings generated for the free-form text fields. A medical report is received via an artificial intelligence engine trained to extract a description of a patient injury, and the description is vectorized into a second embedding vector. The second embedding vector is compared against the stored vector embeddings within the hybrid database, one or more injury categories are selected based on the comparison, and a structured result including at least a predefined label is output.

### CROSS-REFERENCE TO RELATED APPLICATIONS

This application claims the benefit of and priority to both 1) U.S. Provisional Patent Application Ser. No. 63/698,930 filed on 25 Sep. 2024 and entitled “HEALTHCARE MANAGEMENT SYSTEM,” and 2) U.S. Provisional Patent Application Ser. No. 63/750,419 filed on 28 Jan. 2025 and entitled “HEALTHCARE MANAGEMENT SYSTEM.” Each of the aforementioned applications are expressly incorporated herein by reference in their entireties.

### BACKGROUND

Computers and computing systems are increasingly relied upon to manage medical information and assist in clinical decision making. In particular, medical reports such as radiologist notes, imaging summaries, and physical therapy assessments are often generated in natural language, which may include varying terminology, abbreviations, and descriptive phrases for the same underlying medical condition. This variability can make it difficult to consistently classify injuries, generate accurate recovery recommendations, and integrate information into electronic health record systems.

Conventional healthcare information systems typically rely on structured codes such as ICD or CPT codes to categorize patient conditions. While these codes provide a standardized vocabulary, they often require manual entry or human curation. Free-text notes from medical professionals, which frequently contain critical diagnostic details, are therefore underutilized or inconsistently mapped to structured categories.

Accordingly, there is a need for improved systems and methods that enable consistent, structured analysis of medical reports.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

In some embodiments, the techniques described herein relate to a computer system for structured analysis of medical reports including: one or more processors; and one or more computer-readable media having stored thereon executable instructions that when executed by the one or more processors configure the computer system to perform at least the following: receive written text from a user, wherein the written text includes linguistic variants and descriptive phrases associated with different injury categories; vectorize the written text into a first embedding vector, wherein the first embedding vector includes a numerical representation of the written text, expressed within a multi-dimensional semantic vector space; store the written text and the first embedding vector within a hybrid database, wherein the hybrid database includes: a plurality of predefined labels corresponding to injury categories and severity codes, free-form text fields storing the linguistic variants and the descriptive phrases associated with the different injury categories, and stored vector embeddings generated for at least the free-form text fields; receive a medical report from a user via an artificial intelligence engine, wherein the artificial intelligence engine is trained to extract a description of a patient injury from the medical report; vectorize the description of the patient injury into a second embedding vector, wherein the second embedding vector includes a numerical representation of the description of the patient injury, expressed within the multi-dimensional semantic vector space; compare the second embedding vector from the medical report against the stored vector embeddings within the hybrid database; select one or more matching injury categories based on the comparison; and output a structured result including at least a predefined label.

In some embodiments, the techniques described herein relate to a computer-implemented method for structured analysis of medical reports, including: receiving written text from a user, wherein the written text includes linguistic variants and descriptive phrases associated with different injury categories; vectorizing the written text into a first embedding vector, wherein the first embedding vector includes a numerical representation of the written text, expressed within a multi-dimensional semantic vector space; storing the written text and the first embedding vector within a hybrid database, wherein the hybrid database includes: a plurality of predefined labels corresponding to injury categories and severity codes, free-form text fields storing the linguistic variants and the descriptive phrases associated with the different injury categories, and stored vector embeddings generated for at least the free-form text fields; receiving a medical report from a user via an artificial intelligence engine, wherein the artificial intelligence engine is trained to extract a description of a patient injury from the medical report; vectorizing the description of the patient injury into a second embedding vector, wherein the second embedding vector includes a numerical representation of the description of the patient injury, expressed within the multi-dimensional semantic vector space; comparing the second embedding vector from the medical report against the stored vector embeddings within the hybrid database; selecting one or more matching injury categories based on the comparison; and outputting a structured result including at least a predefined label.

In some embodiments, the techniques described herein relate to a computer-readable media including one or more physical computer-readable storage media having stored thereon computer-executable instructions that, when executed at a processor, cause a computer system to perform a method for structured analysis of medical reports, the method including: receiving written text from a user, wherein the written text includes linguistic variants and descriptive phrases associated with different injury categories; vectorizing the written text into a first embedding vector, wherein the first embedding vector includes a numerical representation of the written text, expressed within a multi-dimensional semantic vector space; storing the written text and the first embedding vector within a hybrid database, wherein the hybrid database includes: a plurality of predefined labels corresponding to injury categories and severity codes, free-form text fields storing the linguistic variants and the descriptive phrases associated with the different injury categories, and stored vector embeddings generated for at least the free-form text fields; receiving a medical report from a user via an artificial intelligence engine, wherein the artificial intelligence engine is trained to extract a description of a patient injury from the medical report; vectorizing the description of the patient injury into a second embedding vector, wherein the second embedding vector includes a numerical representation of the description of the patient injury, expressed within the multi-dimensional semantic vector space; comparing the second embedding vector from the medical report against the stored vector embeddings within the hybrid database; selecting one or more matching injury categories based on the comparison; and outputting a structured result including at least a predefined label.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates an embodiment of a computer system for structured analysis of medical reports.

FIG. 2 illustrates an embodiment of a user interface for creating entries within the hybrid database.

FIG. 3 illustrates an example process by which embedding vectors are created from both user-provided text and medical report data, and how those vectors are compared for structured analysis.

FIG. 4 illustrates a flowchart of a steps in a method for structured analysis of medical reports.

### DETAILED DESCRIPTION

Disclosed embodiments relate to a computer system for structured analysis of medical reports. More particularly, the disclosure relates to systems, methods, and computer-readable media for receiving written text from a user, vectorizing the written text into embedding vectors, storing the text and vectors in a hybrid database, and comparing the vectors against medical report data processed by an artificial intelligence engine to produce structured results.

In at least one embodiment, the computer system comprises one or more processors and one or more computer-readable media storing executable instructions that, when executed, cause the system to perform structured analysis of medical reports. The system is configured to receive written text from a user, where the written text may include linguistic variants and descriptive phrases associated with different injury categories. The written text is vectorized into a first embedding vector, the embedding vector comprising a numerical representation of the written text expressed within a multi-dimensional semantic vector space. Both the written text and the first embedding vector may be stored in a hybrid database.

The hybrid database provides a unique data format comprising both predefined labels corresponding to injury categories and severity codes and free-form text fields storing linguistic variants and descriptive phrases associated with the injury categories. In addition, the hybrid database maintains stored vector embeddings generated for at least the free-form text fields. By integrating predefined labels with free-form text, the hybrid database enables flexible storage of structured and unstructured medical descriptions.

The computer system is further configured to receive a medical report from a user via an artificial intelligence engine. The artificial intelligence engine is trained to extract a description of a patient injury from the medical report. The description is vectorized into a second embedding vector, which is likewise expressed in the same multi-dimensional semantic vector space. The system then compares the second embedding vector against stored vector embeddings within the hybrid database to identify similarity. One or more matching injury categories are selected based on the comparison, and the system outputs a structured result comprising at least a predefined label.

By employing embedding vectors, the disclosed embodiments may capture the semantic meaning of both user-entered text and AI-generated text, thereby improving the accuracy of matching descriptive phrases to structured injury categories. The use of a hybrid database allows both structured predefined labels and unstructured free-form descriptions to be preserved and analyzed within the same system. As a result, the computer system supports consistent classification of injuries, enables alignment of narrative text with standardized categories, and facilitates the generation of reliable structured outputs that can be used for clinical decision-making, reporting, or integration with electronic health records.

Existing medical informatics systems face multiple challenges when processing unstructured clinical narratives. Traditional electronic health record (EHR) systems rely heavily on structured codes (e.g., ICD, CPT, SNOMED), which provide standardized classification but require manual coding and do not accommodate the richness or variability of narrative medical text. Natural language processing (NLP) and artificial intelligence (AI) models, particularly large language models (LLMs), have been applied to free-text medical reports to automate extraction of relevant features. However, these approaches suffer from semantic ambiguity and lack of grounding. LLMs are prone to “hallucinations,” where the model generates plausible but incorrect interpretations of input text or introduces information not present in the source. For example, an AI system may infer a “complete ACL tear” when a radiology report only notes “fiber disruption,” thereby overclassifying the injury. Conventional keyword-based or synonym-table approaches do not solve this problem, as they fail to capture nuanced semantic relationships between phrases and often degrade in performance as vocabularies expand.

The technical difficulty arises from the fact that unstructured text embeddings produced by AI models exist in high-dimensional vector space without a stable anchor to canonical medical categories. Without constraints, similarity comparisons or generative outputs can drift toward irrelevant or overly general concepts, especially when embeddings are computed on heterogeneous or noisy text. The present invention addresses this by introducing a hybrid database that stores both structured predefined labels (e.g., injury categories and severity codes) and free-form descriptive text fields, each associated with stored vector embeddings. When an AI engine extracts descriptive text from a medical report and vectorizes it, the system compares that embedding against the controlled set of stored embeddings within the hybrid database. Because each embedding is anchored to a predefined label, the comparison process maps free-form or ambiguous text back to a stable, curated set of categories. This architecture effectively grounds AI outputs in a bounded semantic space, mitigating hallucinations and ensuring that generated structured results always correspond to recognized injury categories. In database terms, the hybrid model functions as both a vector store and a relational schema: free-form embeddings provide semantic flexibility, while predefined labels act as referential integrity constraints, guaranteeing that AI-driven analysis yields reliable, clinically meaningful outputs.

In at least one embodiment, a benefit of the disclosed hybrid database architecture is its ability to mitigate AI hallucinations. When the artificial intelligence engine (described below) produces an extracted description from a medical report, that description is vectorized and compared against the embeddings stored in the hybrid database. Because each embedding is linked to a predefined label, the AI engine's outputs are constrained to resolve within the bounded semantic space defined by the database. This prevents the system from generating unverified or fabricated injury categories, a limitation commonly observed in unconstrained large language model outputs. In effect, the hybrid database serves as a semantic grounding layer, converting probabilistic AI inferences into deterministic classifications.

Another advantage of the hybrid database is its extensibility without schema redesign. New linguistic variants or descriptive phrases can be added directly, at any time, into the free-form text fields (described below) and vectorized into embeddings without altering the predefined label schema. Stated otherwise, in at least one embodiment, the system supports iterative user editing of stored free-form text fields, and re-vectorizes the edited text for database storage. Additionally, in at least one embodiment, only a human user can update the free-form text fields, which may prevent an artificial engine from injecting hallucinations into the free-form text fields or otherwise drifting from the intended meaning associated with a specific field. This allows the hybrid database to evolve organically with medical language, capturing colloquial terms, new radiology shorthand, or emerging clinical terminology, while still mapping all entries back to stable predefined categories. This feature is particularly beneficial in the medical domain, where descriptive language can evolve faster than formal coding systems.

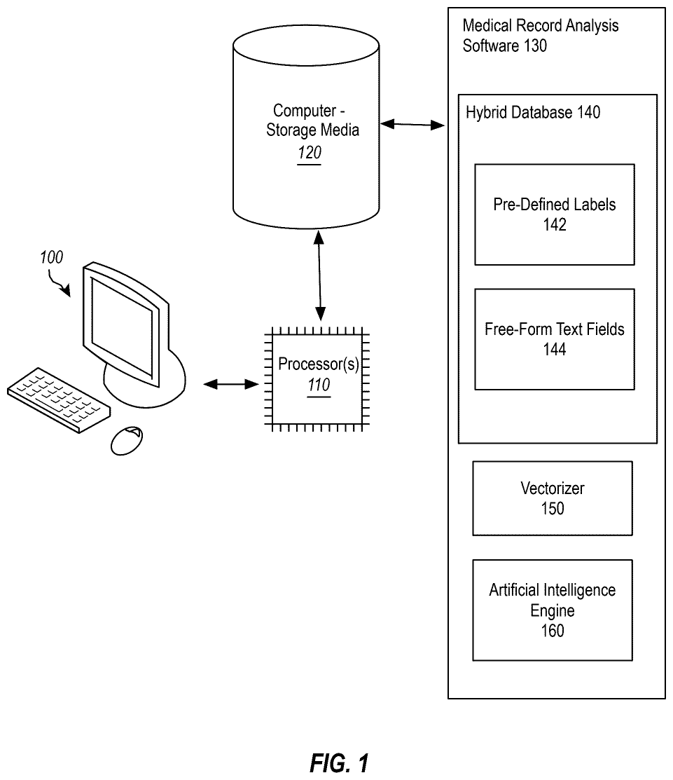

FIG. 1 illustrates an embodiment of a computer system 100 for structured analysis of medical reports. The system comprises one or more processors 110 and one or more computer-readable storage media 120 configured to store and execute medical record analysis software 130. The software may include a hybrid database 140, a vectorizer 150, and an artificial intelligence engine 160.

In at least one embodiment, the hybrid database 140 is structured to include at least predefined labels 142 and free-form text fields 144. The predefined labels 142 correspond to recognized injury categories and severity codes, providing a consistent and standardized schema for classifying medical injuries. In parallel, the free-form text fields 144 allow the system to capture linguistic variants and descriptive phrases that medical professionals may use in unstructured reports. These dual data structures-structured labels and unstructured text—are linked through associated stored vector embeddings. By storing embeddings for both types of entries, the hybrid database 140 serves as an integrated repository that combines the flexibility of vector search with the consistency of structured labels.

The vectorizer 150 transforms incoming text into embedding vectors expressed within a multi-dimensional semantic vector space. When a user provides written text, such as a radiologist note or descriptive phrase, the vectorizer 150 produces a first embedding vector. This vector provides a numerical representation of the semantic content of the written text. The written text and the first embedding vector are then stored within the hybrid database 140, ensuring both the raw language and its semantic representation are preserved for future comparison.

In at least one embodiment, the vectorizer 150 is a machine learning component configured to transform arbitrary written text into a dense embedding vector located in a multi-dimensional semantic vector space. This component allows the computer system 100 to represent linguistic inputs in a numerically tractable form suitable for similarity search and structured comparison.

At a high level, the vectorizer 150 may accept raw written text input, such as a radiologist's written note or an AI-engine extracted injury description, and process the written text through a sequence of NLP operations. As used herein, “written text” comprises text written by hand, text typed by hand, text transcribed through dictation, or any other form of written text information added to a medical record. Initially, the text is tokenized into subword units (e.g., WordPiece or byte-pair encodings). Each token is mapped to an initial embedding vector via a learned embedding matrix E∈ [symbol] ^{V×c}, where V is the vocabulary size and d is the embedding dimension.

The token embeddings may then be passed through a transformer-based neural network encoder, typically consisting of multi-head self-attention layers, feed-forward projections, and residual connections. The encoder contextualizes each token embedding by computing weighted dependencies across the full sequence. For token tᵢ, the contextual embedding is computed as:

$${h}_{i} = TransformerEncoder ( E [ {t}_{1} ] , E [ {t}_{2} ] , … , E [ {t}_{n} ] )$$

where n is the sequence length.

To generate a single fixed-length vector representation for the entire input sequence, the vectorizer 150 may apply a pooling strategy, such as: CLS-token pooling using the embedding of a designated classification token (e.g., [CLS]), Mean pooling by averaging contextualized embeddings across all tokens, or Max pooling by selecting the maximum activation along each dimension.

The vectorizer 150 may be implemented as a fine-tuned variant of a pre-trained model (e.g., BioBERT, ClinicalBERT, or Sentence-BERT), optimized on a domain-specific corpus of medical reports. Fine-tuning ensures that embeddings encode domain-relevant semantics such as injury terminology, synonyms, and context-specific phraseology.

Once produced, the embedding vector is L2-normalized to ensure unit length, enabling direct use of cosine similarity as a distance metric for comparison. For an embedding v, normalization is computed as:

$$\hat{v} = \frac{v}{{‖ v ‖}_{2}}$$

The normalized vector is then persisted to the hybrid database 140, where it can be efficiently indexed using approximate nearest neighbor (ANN) search structures, such as hierarchical navigable small-world (HNSW) graphs or product quantization (PQ).

From a systems perspective, the vectorizer 150 acts as the deterministic bridge between unstructured linguistic inputs and the structured embedding space used for database comparison. By ensuring that both user-entered text (first embedding vector) and AI-engine extracted descriptions (second embedding vector) are encoded within the same semantic space, the vectorizer enables direct vector-space alignment. This consistency allows the computer system 100 to resolve varied clinical language into stable predefined labels.

The computer system 100 is further configured to process a medical report received via the artificial intelligence (AI) engine 160. The AI engine 160 is trained to extract descriptions of patient injuries from radiology reports or similar medical narratives. Once an injury description is extracted, the vectorizer 150 transforms the text into a second embedding vector. This second embedding vector is then passed into a comparison process against the stored vector embeddings within the hybrid database 140.

In at least one embodiment, the AI engine 160 is responsible for transforming raw medical reports into semantically meaningful descriptions of patient injuries that can be aligned with the hybrid database 140. The AI engine 160 may be trained on large corpora of radiology reports, orthopedic clinical notes, and annotated injury datasets, enabling it to recognize domain-specific terminology and extract diagnostically relevant phrases. In operation, the AI engine 160 ingests a full-text report, performs document segmentation to identify sections such as “Findings” and “Impressions,” and applies a natural language understanding pipeline to isolate textual spans that describe anatomical structures, abnormalities, and clinical severity. Unlike generic entity recognition, the AI engine is configured to generate concise descriptive outputs optimized for downstream vectorization.

Once an injury description is extracted, the AI engine 160 normalizes the text by removing redundant modifiers, expanding abbreviations, and mapping shorthand notation into complete medical terms. For example, the phrase “ACL sprn” in a radiologist note may be normalized to “ACL sprain.” This normalization step ensures consistency across extracted text, reducing variance before the text is passed to the vectorizer 150. The engine then outputs the normalized injury description as a structured text string. That output is subsequently vectorized into a second embedding vector, which resides in the same multi-dimensional semantic vector space as the user-provided written text embeddings.

The AI engine 160 is designed not to operate in isolation but to be tightly integrated with the hybrid database 140. This integration addresses the well-documented issue of hallucinations in generative models, where an unconstrained AI might infer or fabricate diagnostic content not actually present in the input report. In this architecture, any injury description produced by the AI engine 160 must be validated against stored embeddings anchored to predefined labels 142 and free-form text fields 144 within the hybrid database. The subsequent comparison step filters the AI's outputs through a similarity search process, ensuring that only injury descriptions with strong semantic alignment to stored embeddings are accepted. In effect, the hybrid database 140 serves as a constraint layer that grounds AI outputs in curated medical categories, mitigating hallucinations and producing reliable, clinically valid classifications.

From an implementation standpoint, the AI engine 160 can be realized as a fine-tuned transformer-based model, such as a BERT or GPT derivative trained on medical language. Its extraction functions may leverage token classification heads for entity recognition, span classification models for anatomical structure detection, or sequence-to-sequence heads for abstractive summarization of diagnostic findings. Because the embeddings generated by the vectorizer 150 are sensitive to context, the AI engine's extraction process ensures that only semantically relevant text is submitted for embedding, avoiding dilution of signal with irrelevant portions of the report. This cooperative interaction between the AI engine 160 and the hybrid database 140 establishes a robust pipeline where generative AI is employed for flexible text understanding, but structured database constraints enforce deterministic, reproducible classification results.

The comparison process begins after the vectorizer 150 generates a first embedding vector for the user-entered written text and a second embedding vector for the description of a patient injury extracted by the artificial intelligence engine 160. Both the first and second embedding vectors are expressed within the same multi-dimensional semantic vector space, which allows them to be directly compared against the stored vector embeddings maintained in the hybrid database 140. Within the hybrid database 140, the embeddings are associated either with predefined labels 142, representing canonical injury categories and severity codes, or with free-form text fields 144, which preserve linguistic variants and descriptive phrases.

In certain embodiments, the hybrid database 140 stores hierarchical predefined labels that are organized into parent injury categories and nested subcategories. For example, a parent category such as “Knee Injury” may include subcategories like “Anterior Cruciate Ligament (ACL) Injury” and “Meniscus Injury”. Within the ACL injury category, further subcategories may distinguish between “Partial Tear” and “Complete Tear,” while the meniscus category may subdivide into “Degenerative Tear” and “Displaced Tear.” By structuring predefined labels in this hierarchical fashion, the hybrid database supports multi-resolution classification, allowing the system to map descriptive phrases not only to a general anatomical region but also to progressively more specific injury types. This hierarchy enhances retrieval and comparison, as embedding vectors generated by the vectorizer 150 can resolve to the appropriate level of granularity depending on similarity thresholds, ensuring both coarse-grained and fine-grained classification within the same database schema.

Examples of injury categories stored in the predefined labels 142 of the hybrid database 140 may include musculoskeletal injuries such as anterior cruciate ligament (ACL) tear, medial meniscus tear, Achilles tendon rupture, rotator cuff injury, and lumbar disc herniation. Each injury category may be associated with one or more severity codes, which provide a standardized representation of clinical significance. For instance, an ACL tear may be classified into severity codes such as partial tear (physical-therapy), complete tear (surgical), or strain (self-management). Similarly, a meniscus injury may include severity codes corresponding to degenerative tear (self-management) versus displaced tear (surgical). The severity codes may be encoded as categorical levels such as surgical, physical-therapy, or self-management, or may be extended into finer-grained tiers (e.g., red/yellow/green indicators) depending on clinical context. By binding free-form descriptive text, such as “ligament fiber disruption” or “cartilage fraying,” to these predefined labels and severity codes, the system ensures that variable narrative inputs are normalized into consistent, structured outputs suitable for decision support and downstream analysis.

For efficiency at scale, the stored embeddings in hybrid database 140 are indexed using approximate nearest neighbor search structures, such as hierarchical navigable small-world graphs, product quantization, or other vector indexing methods, enabling sublinear query times across large embedding sets. When the second embedding vector is submitted as a query, the system executes a nearest-neighbor search to identify embeddings stored in the hybrid database 140 that are positioned close to the query vector in semantic space. Cosine similarity may be used as the primary distance metric, which, after normalization of vectors at the output of vectorizer 150, reduces to a dot product operation between the query embedding and stored embeddings. This permits efficient computation using optimized linear algebra libraries or GPU acceleration.

In at least one embodiment, the computer system 100 applies a predefined similarity threshold to filter results, discarding any matches from free-form text fields 144 or predefined labels 142 that do not exceed the required score. Only embeddings with similarity scores greater than or equal to the threshold are retained as valid candidates. The predefined similarity threshold may be statically defined by the computer system 100 or may be dynamic based upon the type of injuries or type of medical records being processed. The valid matches are then ranked in descending order of similarity, and the computer system 100 selects one or more injury categories associated with the top-ranked embeddings. In configurations where multiple injuries are identified, a highest-severity override rule may be applied, ensuring that the structured result emphasizes the most clinically significant category.

Because each embedding in the hybrid database 140 is anchored to a predefined label 142, the structured result always resolves to a canonical, curated injury category rather than arbitrary free-form text. The structured result may also include additional metadata, such as severity codes, treatment recommendations, or recovery timelines, as associated with the selected predefined label 142. In this manner, the outputs of the artificial intelligence engine 160 are consistently grounded in the stable schema of hybrid database 140, effectively mitigating hallucinations or spurious classifications while ensuring clinically reliable, structured outputs.

Once one or more matches are determined, the system selects the appropriate injury category and produces a structured result. The structured result includes at least a predefined label, which provides a canonical representation of the identified injury. In certain embodiments, the structured result may also include associated metadata, such as severity codes or recovery timelines, retrieved from the hybrid database 140.

Accordingly, in at least one embodiment the hybrid database 140 functions as a semantic grounding layer. Free-form text, which is inherently variable and prone to ambiguity, is vectorized and tied back to structured categories. The AI engine 160, which may otherwise generate hallucinations or inconsistent outputs, is constrained by this database structure. By forcing AI-derived embeddings to resolve against stored embeddings anchored to predefined labels, the system ensures that the AI outputs are mapped to stable, curated categories. This architecture solves the challenge of uncontrolled generative behavior by creating a bounded semantic space in which injury classifications are both flexible and reliable.

In practice, this means that radiologists can continue to use varied language in their reports, while the computer system 100 normalizes those descriptions to a structured schema. Similarly, patients or physical therapists may enter colloquial terms into the computer system 100, which are also vectorized and mapped against the hybrid database 140. The combination of free-form capture, embedding comparison, and structured output provides a robust pipeline for translating heterogeneous medical narratives into consistent, machine-readable results.

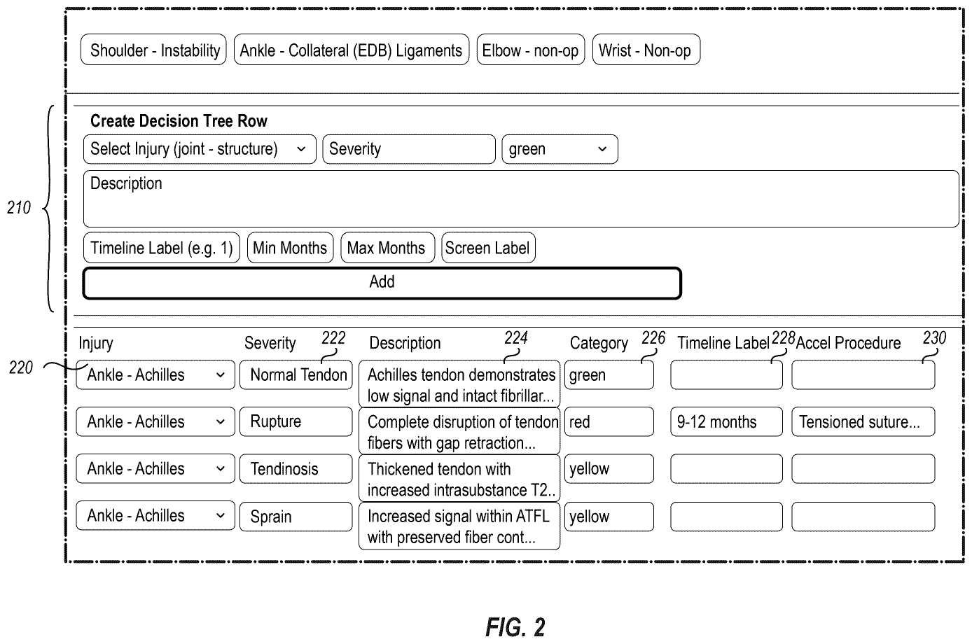

FIG. 2 illustrates a user interface 200 for creating entries within the hybrid database 140. The user interface 200 is configured to allow direct entry of both structured and unstructured information, thereby enabling the construction of database entries that include predefined labels as well as free-form text fields, consistent with the claims.

The user interface 200 includes a create injury portion 210, which comprises a set of predefined label selectors and corresponding free-form text boxes. This dual-input design reflects the structure of the hybrid database 140 described in FIG. 1, which integrates predefined labels 142 with free-form text fields 144. By supporting both input modalities, the create injury portion 210 ensures that canonical identifiers for injuries can be stored alongside narrative or descriptive content authored by clinicians or users.

An example entry in the user interface 200 demonstrates this hybrid representation. In this example, the injury predefined label 220 is “Ankle-Achilles,” which corresponds to a structured label defining the anatomical region and injury category. The entry also includes a severity entry 222, implemented as a free-form text box containing the phrase “Normal Tendon.” This allows nuanced severity descriptors to be captured in natural language, while still being linked to the structured injury category label. A description free-form entry 224 is further provided, containing narrative medical text: “Achilles tendon demonstrates low signal and intact fibrillar pattern with normal thickness”. This text reflects the type of descriptive phrase a radiologist might include in an MRI report, and when vectorized by the vectorizer 150 of FIG. 1, it produces an embedding that can be stored alongside the structured injury category.

The example also includes a category predefined label 226 of “green,” representing a severity code associated with the injury. A variety of different category labels may be used, such as labels that describe severity codes including at least surgical, physical-therapy, and self-management categories. In this embodiment, color-coding (green, yellow, red) provides a concise representation of severity classification, with “green” corresponding to the lowest-severity or self-management tier. Finally, the entry includes an accelerated procedures free-form text entry 230, which reads “tensioned suture+inter . . . ”. This demonstrates how the system allows procedure-specific information to be captured in unstructured form, enabling free-form notes about treatment pathways to be preserved alongside the structured injury category and severity code.

Through the combination of predefined labels 220 and 226 with free-form entries 222, 224, and 230, the user interface 200 enables the hybrid database 140 to maintain a comprehensive representation of injury information. This design ensures that the computer system 100 described in FIG. 1 can store both canonical identifiers and the linguistic variants or descriptive phrases required for accurate semantic matching. When processed through the vectorizer 150, the free-form text entries become embedding vectors that can later be compared against AI-extracted embeddings by the artificial intelligence engine 160, thereby linking narrative clinical descriptions to predefined structured results.

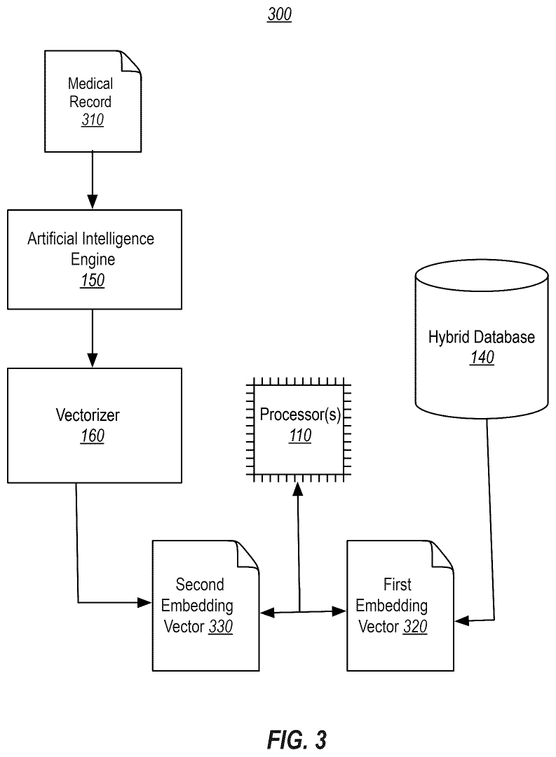

FIG. 3 illustrates an example process 300 by which embedding vectors are created from both user-provided text and medical report data, and how those vectors are compared for structured analysis. In this example, a medical record 310 is provided to the artificial intelligence engine 150, which parses the report and extracts a textual description of a patient injury. This extracted text is then passed to the vectorizer 150, which generates a second embedding vector 330. The second embedding vector 330 is a numerical representation of the injury description, expressed within a multi-dimensional semantic vector space.

In parallel to or prior to the processing of the second embedding vector 330, written text provided by a user, such as narrative phrases, severity descriptions, or annotations, is also processed through the vectorizer 150 to generate a first embedding vector 320. Both the written text and the first embedding vector 320 are stored in the hybrid database 140, which maintains predefined labels and free-form text fields as described in FIG. 1. The hybrid database 140 thus provides the ground-truth semantic anchors for injury classification, with each embedding associated either with a predefined label or with free-form text.

The processor(s) 110 coordinate the comparison operation between the vectors. The second embedding vector 330, generated from the medical record 310, is compared against the first embedding vector 320 and other stored embeddings within the hybrid database 140. This comparison is performed using similarity metrics, such as cosine similarity, which quantify the degree of semantic alignment between the vectors. By applying a threshold to the similarity score, the system ensures that only embeddings with sufficient semantic proximity are identified as valid matches.

The process 300 shown in FIG. 3 demonstrates how the computer system 100 bridges unstructured clinical narratives and structured injury categories. The artificial intelligence engine 150 provides an initial extraction of injury-relevant content from medical record 310, but its outputs are not accepted directly. Instead, the outputs are converted into a second embedding vector 330 and grounded against embeddings in hybrid database 140. This process 300 mitigates hallucinations and variability from the AI engine 150, because the final structured result must resolve to a predefined label in the hybrid database 140. In this manner, process 300 shows how the embedding pipeline enforces semantic consistency and ensures that injury classifications are deterministic, auditable, and anchored to curated medical categories.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

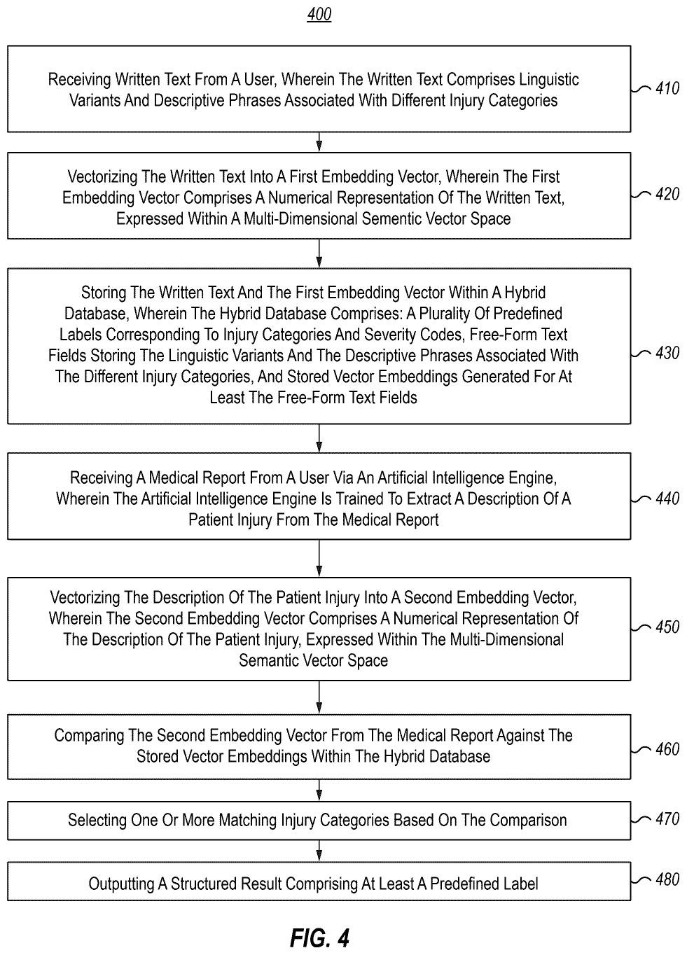

FIG. 4 illustrates a flowchart 400 corresponding to the steps of the method claim. The method begins at step 410, where written text is received from a user. This written text may originate from direct entry via the user interface 200 of FIG. 2, where a user provides linguistic variants, descriptive phrases, or annotations of injuries into the free-form text fields 144. These text entries may provide heterogeneous descriptions, such as “fiber disruption” or “intact fibrillar pattern,” that are not always standardized.

At step 420, the written text is processed by the vectorizer 150 of FIG. 1, which encodes the text into a first embedding vector 320 as illustrated in FIG. 3. The embedding vector represents the semantic meaning of the input text in a high-dimensional vector space (e.g., 768 dimensions for transformer encoders). This transformation enables comparison based on semantic similarity rather than simple keyword overlap, a significant technical improvement over conventional synonym matching.

Step 430 involves storing the written text and its corresponding first embedding vector 320 in the hybrid database 140 of FIG. 1. As described with respect to FIG. 2, each entry may include a structured component such as a predefined label 220 or category predefined label 226, and unstructured components such as severity entry 222, description entry 224, or accelerated procedure entry 230. Together, these fields form a hybrid representation where free-form text and structured categories are linked through their associated embeddings.

At step 440, the computer system 100 receives a medical report 310 via the artificial intelligence engine 160. As shown in FIG. 3, the AI engine extracts a description of a patient injury from the unstructured report and outputs a normalized text string. This extraction may involve identifying diagnostic sentences in the “Findings” section of a radiology report and reducing them to a concise injury description, e.g., “partial ACL tear.”

At step 450, the injury description is passed to the vectorizer 150, producing a second embedding vector 330. This embedding is aligned to the same semantic space as the first embedding vector 320, ensuring interoperability between user-entered text and AI-extracted report content.

At step 460, the processor(s) 110 execute a similarity search that compares the second embedding vector 330 against the set of stored embeddings in the hybrid database 140, including first embedding vectors 320 previously generated from user entries. As described in FIG. 3, this comparison may use cosine similarity to identify embeddings that reside close to the query in semantic space. Approximate nearest neighbor search structures may be employed for efficient retrieval across large-scale embedding sets.

At step 470, one or more injury categories are selected based on the comparison results. Because each stored embedding in the hybrid database 140 is anchored to a predefined label 142, the computer system 100 ensures that the matching process resolves to canonical categories, such as “ACL Tear,” “Meniscus Injury,” or “Achilles Tendinosis.” Where multiple embeddings exceed the similarity threshold, results may be ranked by similarity score, or a highest-severity override rule may be applied to prioritize clinically significant categories.

Finally, at step 480, the computer system 100 outputs a structured result comprising at least the predefined label. In some embodiments, the structured result may include additional metadata from the hybrid database 140, such as severity codes, recovery timelines, or treatment recommendations. This output may be delivered to a clinician portal for validation or directly to a patient-facing interface. By grounding the output in predefined labels 142 and associated metadata, the computer system 100 ensures that all AI-derived inferences are constrained by the hybrid database 140, effectively mitigating hallucinations and enforcing deterministic classification.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

Computing system functionality can be enhanced by a computing systems' ability to be interconnected to other computing systems via network connections. Network connections may include, but are not limited to, connections via wired or wireless Ethernet, cellular connections, or even computer to computer connections through serial, parallel, USB, or other connections. The connections allow a computing system to access services at other computing systems and to quickly and efficiently receive application data from other computing systems.

Interconnection of computing systems has facilitated distributed computing systems, such as so-called “cloud” computing systems. In this description, “cloud computing” may be systems or resources for enabling ubiquitous, convenient, on-demand network access to a shared pool of configurable computing resources (e.g., networks, servers, storage, applications, services, etc.) that can be provisioned and released with reduced management effort or service provider interaction. A cloud model can be composed of various characteristics (e.g., on-demand self-service, broad network access, resource pooling, rapid elasticity, measured service, etc), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Cloud and remote based service applications are prevalent. Such applications are hosted on public and private remote systems such as clouds and usually offer a set of web based services for communicating back and forth with clients.

Many computers are intended to be used by direct user interaction with the computer. As such, computers have input hardware and software user interfaces to facilitate user interaction. For example, a modern general purpose computer may include a keyboard, mouse, touchpad, camera, etc. for allowing a user to input data into the computer. In addition, various software user interfaces may be available.

Examples of software user interfaces include graphical user interfaces, text command line based user interface, function key or hot key user interfaces, and the like.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media.

Physical computer-readable storage media includes RAM, ROM, EEPROM, CD-ROM or other optical disk storage (such as CDs, DVDs, etc), magnetic disk storage or other magnetic storage devices, or any other medium which can be used to store desired program code means in the form of computer-executable instructions or data structures and which can be accessed by a general purpose or special purpose computer. Computer-readable media and computer-storage media herein refer to non-transitory media.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A computer system for structured analysis of medical reports, comprising:
one or more processors; and
one or more computer-readable media having stored thereon executable instructions that when executed by the one or more processors configure the computer system to perform at least the following:
receive written text from a user, wherein the written text comprises linguistic variants and descriptive phrases associated with different injury categories;
vectorize the written text into a first embedding vector, wherein the first embedding vector comprises a numerical representation of the written text, expressed within a multi-dimensional semantic vector space;
store the written text and the first embedding vector within a hybrid database, wherein the hybrid database comprises:
a plurality of predefined labels corresponding to injury categories and severity codes,
free-form text fields storing the linguistic variants and the descriptive phrases associated with the different injury categories, and
stored vector embeddings generated for at least the free-form text fields;
receive a medical report from a user via an artificial intelligence engine, wherein the artificial intelligence engine is trained to extract a description of a patient injury from the medical report;
vectorize the description of the patient injury into a second embedding vector, wherein the second embedding vector comprises a numerical representation of the description of the patient injury, expressed within the multi-dimensional semantic vector space;
compare the second embedding vector from the medical report against the stored vector embeddings within the hybrid database;
select one or more matching injury categories based on the comparison; and
output a structured result comprising at least a predefined label.

2. The computer system of claim 1, wherein the hybrid database stores hierarchical predefined labels organized into parent injury categories and subcategories.

3. The computer system of claim 1, wherein the hybrid database stores severity codes including at least surgical, physical-therapy, and self-management codes.

4. The computer system of claim 1, wherein the written text is vectorized using contextual embeddings generated by a transformer-based neural network.

5. The computer system of claim 1, wherein comparing vectors includes calculating cosine similarity between the second embedding vector and stored embedding vectors.

6. The computer system of claim 1, wherein selection of a matching injury categories requires that similarity exceed a predefined threshold value.

7. The computer system of claim 1, wherein the artificial intelligence engine outputs descriptive text that is discarded if no corresponding stored embedding in the hybrid database is identified.

8. The computer system of claim 1, wherein the hybrid database allows a user to annotate free-form text with severity indicators that are stored alongside embedding vectors.

9. The computer system of claim 1, wherein the computer system supports iterative user editing of stored free-form text fields, and re-vectorizes the edited text for database storage.

10. The computer system of claim 1, wherein multiple matching injury categories are ranked according to similarity scores and the structured result presents a highest-ranked category.

11. The computer system of claim 1, wherein the structured result further comprises a recovery timeline retrieved from the hybrid database.

12. A computer-implemented method for structured analysis of medical reports, comprising:
receiving written text from a user, wherein the written text comprises linguistic variants and descriptive phrases associated with different injury categories;
vectorizing the written text into a first embedding vector, wherein the first embedding vector comprises a numerical representation of the written text, expressed within a multi-dimensional semantic vector space;
storing the written text and the first embedding vector within a hybrid database, wherein the hybrid database comprises:
a plurality of predefined labels corresponding to injury categories and severity codes,
free-form text fields storing the linguistic variants and the descriptive phrases associated with the different injury categories, and
stored vector embeddings generated for at least the free-form text fields;
receiving a medical report from a user via an artificial intelligence engine, wherein the artificial intelligence engine is trained to extract a description of a patient injury from the medical report;
vectorizing the description of the patient injury into a second embedding vector, wherein the second embedding vector comprises a numerical representation of the description of the patient injury, expressed within the multi-dimensional semantic vector space;
comparing the second embedding vector from the medical report against the stored vector embeddings within the hybrid database;
selecting one or more matching injury categories based on the comparison; and
outputting a structured result comprising at least a predefined label.

13. The computer-implemented method of claim 12, wherein the hybrid database stores hierarchical predefined labels organized into parent injury categories and subcategories.

14. The computer-implemented method of claim 12, wherein the hybrid database stores severity codes including at least surgical, physical-therapy, and self-management codes.

15. Computer-implemented method of claim 12, wherein the written text is vectorized using contextual embeddings generated by a transformer-based neural network.

16. The computer-implemented method of claim 12, wherein comparing vectors includes calculating cosine similarity between the second embedding vector and stored embedding vectors.

17. The computer-implemented method of claim 12, wherein selection of a matching injury categories requires that similarity exceed a predefined threshold value.

18. The computer-implemented method of claim 12, wherein the hybrid database allows a user to annotate free-form text with severity indicators that are stored alongside embedding vectors.

19. The computer system of claim 1, wherein the computer system supports iterative user editing of stored free-form text fields, and re-vectorizes the edited text for database storage.

20. A computer-readable media comprising one or more physical computer-readable storage media having stored thereon computer-executable instructions that, when executed at a processor, cause a computer system to perform a method for structured analysis of medical reports, the method comprising:
receiving written text from a user, wherein the written text comprises linguistic variants and descriptive phrases associated with different injury categories;
vectorizing the written text into a first embedding vector, wherein the first embedding vector comprises a numerical representation of the written text, expressed within a multi-dimensional semantic vector space;
storing the written text and the first embedding vector within a hybrid database, wherein the hybrid database comprises:
a plurality of predefined labels corresponding to injury categories and severity codes,
free-form text fields storing the linguistic variants and the descriptive phrases associated with the different injury categories, and
stored vector embeddings generated for at least the free-form text fields;
receiving a medical report from a user via an artificial intelligence engine, wherein the artificial intelligence engine is trained to extract a description of a patient injury from the medical report;
vectorizing the description of the patient injury into a second embedding vector, wherein the second embedding vector comprises a numerical representation of the description of the patient injury, expressed within the multi-dimensional semantic vector space;
comparing the second embedding vector from the medical report against the stored vector embeddings within the hybrid database;
selecting one or more matching injury categories based on the comparison; and
outputting a structured result comprising at least a predefined label.
